SEQUENCE LISTING (10 sequences)

SEQ ID NO: 1 - DNA, 126 bp, Homo sapiens
  CDS (1)..(126)
tacgcggaagggactttcatcagtgactacagtattgccatggacaagattcaccaacaagactttgtgaactggctgctggcccaaaaggggaagaagaatgactggaaacacaacatcacccag

SEQ ID NO: 2 - protein, 42 aa, Homo sapiens
YAEGTFISDYSIAMDKIHQQDFVNWLLAQKGKKNDWKHNITQ

SEQ ID NO: 3 - DNA, 603 bp, Rattus sp.
  CDS (176)..(301)
ggaagagctaagaagagctgttggctcggggacacaatctaggaagatggtggctttgaagacctgctctctgctgctggtgctcctgttcctggctgtcgggctgggagaaaaagaagaggttgagttccgatcccatgctaaatttgctggcccccgaccacgaggcccaaggtatgcagaggggactttcatcagtgattacagcatcgccatggacaagatccgccaacaagactttgtgaactggcttctggcccagaaggggaagaagaatgactggaaacacaacctcacccagagagaggcccgggctttggagctggcaggacaatctcagagaaacgaggagaaagaggcacaggggagctctttgcccaagagcctcagtgatgaagatgtgctgagggaccttctgattcaagagctactggcctggatggcggaccaagcagagctctgtcgactgaggtcccagtgaccgacctaccccggagcaggactggactctgaccttagcttgctcagatcctgcttctgccctggtccaaagtctctgagagaaccaaaccaataaagccttgagctgaagtaaaaaaaaaa

SEQ ID NO: 4 - protein, 42 aa, Rattus sp.
YAEGTFISDYSIAMDKIRQQDFVNWLLAQKGKKNDWKHNLTQ

SEQ ID NO: 5 - DNA, 25 bp, Artificial Sequence
  synthetic construct
aagaggttgagttccgatcccatgc

SEQ ID NO: 6 - DNA, 24 bp, Artificial Sequence
  synthetic construct
gattgtcctgccagctccaaagcc

SEQ ID NO: 7 - DNA, 48 bp, Artificial Sequence
  synthetic construct
ggctttggagctggcaggacaatctcagagaaacgaggagaaagaggc

SEQ ID NO: 8 - DNA, 48 bp, Artificial Sequence
  synthetic construct
tgctggcccccgaccacgaggcccaaggtatgcagaggggactttcat

SEQ ID NO: 9 - DNA, 48 bp, Artificial Sequence
  synthetic construct
gtacaggtgagcactgacttgggctgaagctcaagagttggttctgcc

SEQ ID NO: 10 - DNA, 48 bp, Artificial Sequence
  synthetic construct
cctgttcacgtctttcatgctgcgagcaggggccatcctcacccgaga

(19) United States
(12) Patent Application Publication
(10) Pub. No.: US 2006/0084597 A2
(43) Pub. Date: Apr. 20, 2006

(54) USE OF COMPOUNDS HAVING GIP ACTIVITY FOR THE TREATMENT OF DISORDERS ASSOCIATED WITH ABNORMAL LOSS OF CELLS AND/OR FOR THE TREATMENT OF OBESITY

(75) Inventors: Jenny Nyberg, Goteborg (SE); Peter Eriksson, Goteborg (Sweden)

Correspondence Address: DARBY & DARBY P.C., P. O. BOX 5257, NEW YORK, NY, 10150-5257, US

(73) Assignee: Eisai Co., Ltd., Tokyo (JP)

(21) Appl. No.: 10/518,159

(22) Filed: Jan. 18, 2005

Related U.S. Application Data
(63) Continuation of application No. 60/387,390, filed on Jun. 11, 2002.
Application No. 60/387,390 is a continuation of application No. 0201783-8, filed on Jun. 11, 2002.

(30) Foreign Application Priority Data
Jun. 11, 2002 (SE) 0201783-8

(51) Int. Cl. A61K 38/17

(52) U.S. Cl. 514/12

(57) ABSTRACT

Abstract of the Disclosure

Use of a compound having 50% activity or more of the activity of gastric inhibitory polypeptide, GIP, when tested in the same assay under the same conditions, and/or the use of GIP, analogues and fragments thereof, for the manufacture of a pharmaceutical composition for prophylaxis and/or treatment of conditions caused or characterized by abnormal loss of cells and/or for prophylaxis and/or treatment of over weight and obesity. A compound as described above for the prophylaxis and/or treatment of over weight and obesity. Use of antagonists to GIP or the GIP receptor for the prophylaxis and/or treatment of disorders caused or characterized by hyperproliferation of cells and/or abnormally low body weight. A compound for the manufacture of a composition for prophylaxis and/or treatment of depression and/or mood disorders, and a method for determining the level of GIP in the brain of a mammal, is also included.

### Detailed Description of the Invention

### CROSS-REFERENCE TO PRIOR APPLICATION

This is a U.S. national phase application under 35 U.S.C. §371 of International Patent Application No. PCT/EP2003/006207 filed June 11, 2003, and claims the benefit of Swedish Patent Application No. 0201783-8 filed June 11, 2002 and U.S. Patent Application No. 60/387,390 both of which are incorporated by reference herein. The International Application was published in English on December 18, 2003 as WO 03/103697 A2 and A3 under PCT Article 21(2).

### FIELD OF THE INVENTION

The present invention relates to the use of a compound having at least 50% activity of the activity of GIP when tested in the same assay under the same conditions, and/or the use of GIP, analogues and fragments thereof, for the manufacture of a pharmaceutical composition for prophylaxis and/or treatment of conditions caused or characterized by abnormal loss of cells. The invention also relates to a compound as described above for the prophylaxis and/or treatment of over weight and obesity.

### BACKGROUND OF THE INVENTION

Traumatic, asphyxial, hypoxic, ischemic, toxic, infectious, degenerative or metabolic insults to the central nervous system (CNS) often result in damages to several different cell types. Thus, damages to the brain by trauma, asphyxia, toxins, ischemia or infections frequently cause neurological and cognitive deficits.

Perhaps the most severe form of neurodegeneration is that seen after stroke. This form of cerebral ischemia results in the death of neurons, as well as glial cells and vascular elements of the brain. Quite often a stroke results in paralysis, memory loss, and an inability to communicate.

Another form of cerebral ischemia that can be quite devastating to important groups of selectively vulnerable neurons, is global ischemia. Global cerebral ischemia is commonly seen in victims of cardiac arrest during the period of time the heart is undergoing fibrillation. Neuronal death from global ischemia is a common occurrence in heart attack victims that undergo cardiac arrest and cardiac arrest is a common occurrence in heart attack patients.

Parkinson’s disease is a movement disorder in which symptomatology is defined by three cardinal symptoms; tremor at rest, rigidity and akinesia (Fahn, 1989). The disease often causes loss of specific populations of cells and is in particular associated with the specific loss of dopaminergic neurons in the Substantia nigra.The course of the disease is a progressive one. For a long time, anticholinergic drugs were the only effective treatment of parkinsonian symptoms. The beneficial effect of L-3,4-dihydrophenylalanine (L-DOPA) therapy has increased patient's life expectancy to a significant degree. However, the advanced stage of the disease is dominated by the complications of L-DOPA therapy and lack of L-DOPA responsiveness. A limiting factor in PD therapy is the psychotic potential of many anti-parkinsonian drugs.

Amyotrophic lateral sclerosis, (ALS), is a chronic progressive degenerative disorder, which, in its classical form, appears sporadically. The most prominent pathological change in ALS patients is a loss of large motoreurons in the motor cortex, brain stem and spinal cord. In motoreuron disease, (e.g. ALS), a degeneration of the central pyramidal, the peripheral motor system or both is the reason for the clinical picture.

Another illustration of a degenerative disorder caused by selective loss of a specialized type of neurons is Alzheimer’s disease, (AD), which is associated with loss of cholinergic neurons. Cognitive decline is the essential clinical criteria for AD manifested by memory loss, disorientation and the concomitant loss of enjoyment of life associated therewith. Only after death can the diagnosis be confirmed pathologically by the presence of numerous amyloid and neuritic plaques in the brain.

Similarly, multiple sclerosis, (MS), is associated with loss of myelin and oligodendrocytes. Additionally, there are many other instances in which CNS injuries or diseases can cause damage to oligodendroglia, astroglia, or neuronal cells.

At present, the pharmacological therapy of neurodegenerative disorders is limited to symptomatic treatments that do not alter the course of the underlying disease.

Meanwhile, because of the current dissatisfaction with the currently marketed treatments for the above-described indications within the affected population, the need continues for safer and better treatments which will either slow the process of neurodegeneration associated with complications or conditions such as focal or global ischemia, ALS, Alzheimer’s and Parkinson’s disease or even prevent such neurodegeneration altogether.

### DESCRIPTION OF THE INVENTION

The present invention relates to use of a compound that when tested in an in vitro proliferation assay has an activity that corresponds to at least about 50% of the activity of SEQ ID NO 2 when tested in the same assay under the same conditions, for the manufacture of a pharmaceutical composition for prophylaxis and/or treatment of conditions caused or characterized by abnormal loss of cells. The sequence having SEQ ID NO 2 is the human gastric inhibitory polypeptide GIP. The in vitro proliferation assay may be performed as described below in the Examples, using a CyQUANT Cell Proliferation Kit (Molecular Probes, Eugene, OR), but any other suitable commercially available proliferation assay may of course also be used.

The present inventors have shown the presence of GIP expression and GIP immunoreactivity in the brain. Moreover, they have demonstrated that exogenously delivered GIP induced proliferation of adult-derived hippocampal progenitors in vitro as well as in vivo. Since GIP can cause stem cells, progenitor-cells and other cells, especially cells derived from the central nervous system with the potential to generate differentiated cells, such as neurons, astrocytes and/or oligodendrocytes, to proliferate, GIP may therefore be an important regulatory molecule for neural progenitor cell proliferation in the adult mammalian brain.

Gastric Inhibitory polypeptide (GIP) is an insulinotrophic peptide naturally occurring in human neuroendocrine cells of the small intestine (Buchan A., Polak J., Capella C., Solcia E. and Pearse A., Histochemistry 56: 37-44 (1978)). Its primary function is as an incretin, mediating postprandial insulin release from pancreas (Pederson R., Schubert H. and Brown J., Diabetes 24: 1050-1056 (1975)); Pederson R. and Brown J., Endocrinology 99: 780-785 (1976)).

GIP is a 42 amino acid polypeptide chemically related and showing a structural homology to other members of the secretin-glucagon family of gastrointestinal regulatory polypeptides, including secretin, glucagon, glucagon-like peptide 1 and 2 (GLP 1 and 2), vasoactive intestinal polypeptide (VIP), peptide histidine isoleucine (PHI), growth hormone releasing hormone (GHRH) and pituitary adenylyl cyclase-activating polypeptide (PACAP) (Tseng C., Jarboe L., Landau S., Williams E. and Wolfe M., Proc Natl Acad Sci USA 90: 1992-1996 (1993).

The 42 amino acid gastric inhibitory peptide (GIP) has, apart from its principal insulinotrophic effect on pancreas, been reported to have an influence on other systems. It influences, among others, properties of hepatic venous flow have effects on arteries, enhances collagen synthesis in osteoblast-like cells and increases fatty acid synthesis in adipose tissue

Expression of mRNA for the GIP receptor has been reported in the areas of the brain, including hippocampus and olfactory bulb (Usdin T., Mezey E., Button D., Brownstein M. and Bonner T., Endocrinonogy 133: 2861-2870 (1993); Kaplan A. and Vigna S., Peptides 15: 297-302 (1994)), but the present inventors are first to demonstrate the presence of the GIP peptide itself in the brain. As shown in the Examples, the inventors have used a special antigen retrieval method to detect GIP with immunolabeling and have developed very efficient primers for GIP.

Before going into further details of the invention, in the following is given a list of specific terms used in the present text.

Definitions

Unless defined otherwise, all technical and scientific terms used herein have the same meanings as commonly understood by one of ordinary skill in the art to which this invention belongs.

As used herein, the terms “GIP” or “gastric inhibitory peptide” are meant to refer to the polypeptide having SEQ ID NO 2.

As used herein, the terms “GIP-activity” or “GIP-like activity” relate to the activity of GIP which induces cell-proliferation, and/or the activity which reduces weight gain.

The term “antagonistic” effect as used herein, is meant that the effect is to counter the proliferative effect of GIP on cells, or alternatively, to counter the weight reducing effect of GIP, (i.e. inducing weight gain).

As defined herein, the terms “similarity” or “similar substitutions” mean that chemically similar amino acids replace each other. For example, the basic residues Lys and Arg are considered chemically similar and often replace each other, as do the acidic residues Asp and Glu, the hydroxyl residues Ser and Thr, the aromatic residues Tyr, Phe and Trp, and the non-polar residues Ala, Val, Ile, Leu and Met. Similarity is measured by dividing the number of similar residues by the total number of residues and multiplying the product by 100 to achieve a percentage.

By “identity” is meant a property of sequences that measures their similarity or relationship. Identity is measured by dividing the number of identical residues by the total number of residues and multiplying the product by 100 to achieve a percentage. Thus, two copies of exactly the same sequence have 100% identity, but sequences that are less highly conserved and have deletions, additions, or replacements may have a lower degree of identity. Those skilled in the art will recognize that several computer programs, such as those that employ algorithms such as BLAST (Basic Local Alignment Search Tool, Altschul et al. (1993) J. Mol. Biol. 215:403-410) are available for determining sequence identity.

As defined herein, the term “analogue”, in the context of the GIP polypeptide, is meant a polypeptide in which one or more amino acids are replaced by a different, natural or artificial, amino acid. Also included are variants of GIP in which deletions, substitutions, additions or repeats of one or more amino acids have been introduced. Furthermore, fragments of the peptide, or oligomers of these fragments are included.

The term “neuroprotective” refers to the effect of reducing, arresting or ameliorating nervous insult, and protecting, resuscitating, or reviving nervous tissue that has suffered nervous insult.

As defined herein, the term “abnormal and/or pathological degeneration” refers to a loss of ability and/or loss of control of regeneration of; a differentiated cell and/or tissue, an embryonic stem cell, an adult stem cell, a progenitor cell and/or a cell derived from a stem cell or progenitor cell.

The term “ischemia” refers to localized tissue anemia due to obstruction of the inflow of arterial blood. Global ischemia occurs when blood flow to the entire brain ceases for a period of time. Global ischemia may result from cardiac arrest. Focal ischemia occurs when a portion of the brain is deprived of its normal blood supply. Focal ischemia may result from thromboembolytic occlusion of a cerebral vessel, traumatic head injury, edema or brain tumor. Even if transient, both global and focal ischemia can cause widespread neuronal damage. Although nerve tissue damage occurs over hours or even days following the onset of ischemia, some permanent nerve tissue damage may develop in the initial minutes following the cessation of blood flow to the brain.

The term “neurodegenerative diseases” includes Alzheimer's disease, Parkinson’s disease and diseases that result from ischemia and reperfusion injury and includes neurotoxicity, such as seen in vascular stroke and global and focal ischemia, as well as retinal ischemia.

The term “nervous insult” refers to any damage to nervous tissue and any disability or death resulting therefrom. The cause of nervous insult may be metabolic, toxic, neurotoxic, iatrogenic, thermal or chemical, and includes without limitation, ischemia, hypoxia, cerebrovascular accident, trauma, surgery, pressure, mass effect, hemmorrhage, radiation, vasospasm, neurodegenerative disease, infection, Parkinson's disease, amyotrophic lateral sclerosis (ALS), myelination/demyelination process, epilepsy, cognitive disorder, glutamate abnormality and secondary effects thereof.

The term “preventing neurodegeneration” includes the ability to prevent neurodegeneration in patients diagnosed with a neurodegenerative disease or who are at risk of developing a neurodegenerative disease. The term also encompasses preventing further neurodegeneration in patients who are already suffering from or have symptoms of a neurodegenerative disease.

The terms “treating”, “treat”, or “treatment” refer to: preventing a disease, disorder or condition from occurring in an animal that may be predisposed to the disease, disorder and/or condition, but has not yet been diagnosed as having it; inhibiting the disease, disorder or condition, i.e., arresting its development; and relieving the disease, disorder or condition, i.e., causing regression of the disease, disorder and/or condition.

As defined herein, the term “hyperproliferation” is meant that cells are proliferating faster than usual. Hyperproliferation may result in cancer or tumors, or other diseases such as psoriasis or acne. These diseases are well known and a person skilled in the art will properly be able to diagnose a patient suffering from any of these diseases.

As defined herein, the term “obesity” is defined as the condition in which a patient has a body mass index (BMI, calculated as weight (in kg)/(length (in m))² (kg/m²))above 30.

As defined herein, the term “overweight”is intended to indicate a BMI in a range from about 25 to about 29.9.

The term "abnormal or pathological loss and/or gain of cells" is in the present context used to describe the common technical feature of a wide variety of medical conditions and disorders. The described conditions and disorders are hereby characterized by displaying pathological degeneration of, loss of ability of regeneration of and/or loss of control of regeneration of a differentiated cell and/or tissue, an embryonic stem cell, an adult stem cell, a progenitor cell and/or a cell derived from a stem cell or progenitor cell.

A patient with a BMI below 16 is considered to be anorexic or grossly underweight and may be treated with an antagonist of GIP with the purpose of inducing weight gain.

As used herein the term "mammal" is meant to refer to any mammal, including, for example, primates such as humans and monkeys. Examples of other mammals comprised herein are rabbits, dogs, cats, and livestock such as cattle, goats, sheep and horses.

Abnormal cell loss/gain

The present invention relates to use of a compound as described above for the treatment of any pathological condition affecting abnormal gain and/or loss of differentiated cells or tissues and/or loss of control of proliferation of cells, i.e. of chondrocytes, cardiomyocytes, oligodendroglia, astroglia, neuronal cells, different types of epithelium, endothelium, skin, blood, liver, kidney, bone, pancreatic cells such as pancreatic b-cells, connective tissue, lung tissue, exocrine gland tissue and/or endocrine gland tissue.

The invention relates to use of compounds for the preparation of a medicament for the treatment, including veterinary treatment of livestock, of conditions that are characterized by a abnormal loss of cells, such as Parkinson's disease (which affects dopaminergic neurones), Alzheimer's disease (affecting cholinergic neurones), stroke (affecting neurones and glial cells), multiple sclerosis (affecting oligodendrocytes), asphyxia or hypoxia (affecting neurones and glial cells), epilepsy, heart failure (affecting cardiomyocytes), heart infarction (affecting cardiomyocytes), diabetes (affecting pancreatic beta cells), artrosis or arthritis (affecting chondrocytes), skin disease and burn injuries (affecting dermis and epidermis), liver diseases or failure (affecting hepatocytes), muscle diseases or damages (affecting myocytes), cancer (affecting tissues affected by cancer), pancreatic dysfunction (affecting exocrine or endocrine pancreatic cells such as pancreatic b-cells), inflammatory bowel disease (affecting intestinal cells). Also included in the group of diseases are diseases caused by prions, such as Creutzfeld-Jacob, scrapie and bovine spongiform encefalitis.

The abnormal loss of cells may be caused by traumatic, asphyxial, hypoxic, ischemic, toxic, infectious, degenerative or metabolic insults.

In a specific embodiment of the invention, the abnormal loss of cells may be a degeneration and/or loss of neuronal cells, astrocytes or oligodendrocytes.

In a further embodiment of the invention the abnormal loss of cells is caused by insults to the central or peripheral nervous system, resulting in neurological and/or cognitive deficits.

The conditions to be prevented or treated belongs to the group of Parkinson's disease, Alzheimer's disease, stroke, multiple sclerosis, amyotrophic lateral sclerosis, asphyxia or hypoxia, epilepsy, and diseases caused by prions, such as Creutzfeld-Jacob’s disease, scrapie and bovine spongiform encephalitis (BSE).

The invention relates to use of compounds having an activity when tested in an in vitro proliferation assay, that corresponds to at least about 55%, such as, e.g., at least about 60%, at least about 65%, at least about 70%, at least about 75%, at least about 80%, at least about 85%, at least about 90%, at least about 92%, at least about 94%, at least about 96%, at least about 98% or at least about 99% of the activity of SEQ ID NO 2.

The invention also relates to use of compounds having the same or a higher activity than the compound having SEQ ID NO 2 (GIP), i.e. the invention also relates to use of compounds having an activity that corresponds to at least about 100%, such as, e.g., at least about 110%, at least about 120%, at least about 130%, at least about 140%, at least about 150%, at least about 160%, at least about 170%, at least about 180%, at least about 190%, or at least about 200% of the activity of SEQ ID NO 2.

In one embodiment the invention relates to use according to the invention, wherein the compound is identical to SEQ ID NO 2, i.e. to the active part of human GIP.

The invention also relates to use as described herein, wherein the compound is similar to SEQ ID NO 2, i.e. the compound may have a sequence wherein one or more amino acids of SEQ ID NO 2 are exchanged with chemically similar amino acids.

As is the case with many prophylactic and therapeutic polypeptides, a specific part of the peptide may be responsible for the activity. Consequently, fragments of the peptide are also believed to be within the scope of the invention. Furthermore, dimers, trimers, tetramers, pentamers or other oligomers of these fragments are within the scope of the invention. Additionally, oligomers of the whole gastric inhibitory peptide are within the scope of the invention. Furthermore, analogues wherein the GIP polypeptide has been altered by introduction of deletions, substitutions, additions or repeats of one or more amino acids are also within the scope of the invention.

Accordingly, the invention also relates to a use as described herein, wherein the compound has an identity corresponding to at least about 75% such as, e.g., at least about 80%, at least about 85%, at least about 90%, at least about 95%, at least about 96%, at least about 97%, at least about 98% or at least about 99% to SEQ ID NO 2.

Furthermore, the invention relates to a use as described herein, wherein the compound has a similarity corresponding to at least about 75% such as, e.g., at least about 80%, at least about 85%, at least about 90%, at least about 95%, at least about 96%, at least about 97%, at least about 98% or at least about 99% to SEQ ID NO 2.

In another aspect the invention relates to a compound that – when tested in an in vitro proliferation assay - has an activity that corresponds to at least about 50% of the activity of SEQ ID NO 2 when tested in the same assay under the same conditions with the proviso that the compound is not SEQ ID NO 2 or basic fibroblast growth factor bFGF.

To the best of our knowledge, these two compounds are the only known compounds that fulfill the above criteria, but if other prior art compounds should exist, they should also be excluded from the invention.

The invention also relates to a compound as described for medicinal use. More specific, the compound may be used in the prophylaxis and/or treatment of conditions caused by abnormal loss of cells.

The details and particulars described above relating to the use of a compound according to the invention apply mutatis mutandis to the compounds according to the invention.

The invention also relates to a method of prophylaxis and/or treatment of conditions caused or characterized by an abnormal loss of cells, the method comprising administering a pharmaceutical composition comprising a compound according to invention to a subject in need thereof.

An antagonist directed against GIP, or an antagonist to the GIP receptor will have the reverse effect of the GIP compound. As one of the effects of GIP is stimulation of cell proliferation, an antagonist to GIP or the GIP receptor will most likely have an effect in inhibiting cell proliferation. Accordingly, an antagonist to GIP and/or an antagonist to the GIP receptor may be used in the prophylaxis and/or treatment of diseases or disorders characterized by hyperproliferation of cells.

Thus, the invention relates to the use of an antagonist to GIP for the prophylaxis and/or treatment of conditions caused or characterized by hyperproliferation of cells. The term “antagonists to GIP” describes compounds that bind to the GIP compound, thereby preventing it from binding to the GIP receptor. An example of such a compound may be an antibody towards GIP. The antibody may be a monoclonal antibody, a polyclonal antibody, or a fragment, homologue or analogue thereof. Also, the antibody may be a chimeric, human or humanized antibody. It is aimed that the antibodies produced do not lead to inappropriate immune reactions when administered to a mammal.

The invention also relates to the use of an antagonist to the GIP receptor for the preparation of a pharmaceutical composition for the prophylaxis and/or treatment of conditions caused or characterized by hyperproliferation of cells. Antagonists to the GIP receptor are compounds that interact with the GIP receptor, thereby decreasing the functional activity of the receptor either by inhibiting the action of an agonist or by its own intrinsic activity.

Conditions caused or characterized by hyperproliferation of cells, and which may be prevented or treated by the administration of an antagonist according to the invention, may be selected from the group of neoplastic or cancer diseases such as, e.g., melanoma, non-small-cell lung cancer, small-cell lung cancer, lung cancer, hepatocarcinoma, retioblastoma, astrocytoma, glioblastoma, leukemia, neuroblastoma, pre-neoplastic lesions such as adenomatous hyperplasia and prostatic intraepithelial neoplasia, carcinoma in situ and cancer in the gum, tongue, head, neck, breast, pancreas, prostate, kidney, liver, bone, thyroid, testicle, ovary, mesothelia, cervix, gastrointestinal tract, lymphoma, brain, colon, sarcoma and bladder.

Examples of other diseases to be prevented or treated by the administration of an antagonist according to the invention are tumor-associated diseases, rheumatoid arthritis, inflammatory bowel disease, osteoarthritis, leiomyomas, adenomas, lipomas. hemagioomas, fibromas, vascular occlusion, retenosis, atherosclerosis, oral hairy leukoplasia, benign prostatic hyperplasia, or psoriasis.

Abnormal body weight disorders

Obesity is associated with numerous health implications, which range from non-fatal debilitating conditions such as osteoarthritis, to life-threatening chronic diseases such as coronary heart disease, diabetes type II and certain types of cancer. The physiological consequences of obesity can range from lowered self-esteem to clinical depression. Obesity prevalence is increasing in both developed and undeveloped countries in an epidemic fashion. Since dietary therapy often has a low success rate in the long run, there has been an increasing demand for pharmaceutical alternatives.

It has previously been described that compounds inhibiting GIP will have an anti obesity effect. Contrary to this knowledge the present inventors have surprisingly shown that intracerebroventricular administration of a compound with SEQ ID NO 2 or 4 has an activity in reducing weight gain.

Accordingly, the present invention relates to the use of a compound that when tested in an assay as described in Example 9, wherein rats are given the compound intraventricularly in the brain, followed by the recordation of the weight of each rat has an activity in reducing weight gain that corresponds to at least about 50% of the activity of SEQ ID NO 2 or SEQ ID NO 4 when tested in the same assay under the same conditions using a compound having SEQ ID NO 2 or SEQ ID NO 4 as a control, for the manufacture of a pharmaceutical composition for the prophylaxis or treatment of overweight and/or obesity.

The intracerebroventricular administration of GIP or a compound having GIP activity probably activates GIP receptors within the central nervous system. Especially neurons in the hypothalamus are suspected to be targeted by this administration, but the exact mechanism and target remains uncertain. However, since the effect of GIP seem to be exerted in the brain, a compound according to the invention have to be administered directly to the brain or have to be able to cross the blood brain barrier. Thus, the invention also relates to the use of compounds capable of crossing the blood brain barrier. In a specific example, the invention relates to use of a compound as described herein for the manufacture of a pharmaceutical composition, wherein the pharmaceutical composition further comprises a carrier allowing the transport of the compound across the blood brain barrier.

The invention relates to use of a compound according to the invention, wherein the compound has an activity that corresponds to at least about 55%, such as, e.g., at least about 60%, at least about 65%, at least about 70%, at least about 75%, at least about 80%, at least about 85%, at least about 90%, at least about 92%, at least about 94%, at least about 96%, at least about 98% or at least about 99% of the activity of SEQ ID NO 2 or SEQ ID NO 4.

The invention also relates to use of compounds having the same or a higher activity than compounds having SEQ ID NO 2 or SEQ ID NO 4 (GIP), i.e. the invention also relates to the use of compounds having an activity that corresponds to at least about 100%, such as, e.g., at least about 110%, at least about 120%, at least about 130%, at least about 140%, at least about 150%, at least about 160%, at least about 170%, at least about 180%, at least about 190%, or at least about 200% of the activity of SEQ ID NO 2 or SEQ ID NO 4.

In one embodiment the invention relates to use according to the invention, wherein the compound is identical to SEQ ID NO 2 or SEQ ID NO 4, i.e. to the active part of human GIP.

The invention also relates to use as described herein, wherein the compound is similar to SEQ ID NO 2 or SEQ ID NO 4, i.e. the compound may have a sequence wherein one or more amino acids of SEQ ID NO 2 or SEQ ID NO 4 are exchanged with chemically similar amino acids.

As described above the invention also relates to analogues, fragments and oligomers of the GIP polypeptide.

Accordingly, the invention relates to a use of a compound for the prevention and/or treatment of over weight and obesity as described herein, wherein the compound has an identity corresponding to at least about 75% such as, e.g., at least about 80%, at least about 85%, at least about 90%, at least about 95%, at least about 96%, at least about 97%, at least about 98% or at least about 99% to SEQ ID NO 2.

Furthermore, the invention relates to a use as described herein, wherein the compound has a similarity corresponding to at least about 75% such as, e.g., at least about 80%, at least about 85%, at least about 90%, at least about 95%, at least about 96%, at least about 97%, at least about 98% or at least about 99% to SEQ ID NO 2.

In another aspect the invention relates to a compound that when tested in an assay as described in Example 9, wherein rats are given the compound or a compound having SEQ ID NO 2 or SEQ ID NO 4 intraventricularly in the brain, followed by the recordation of the weight of each rat has an activity in reducing weight gain that corresponds to at least about 50% of the activity of SEQ ID NO 2 or SEQ ID NO 4 when tested in the same assay under the same conditions. Any prior art compound that fulfills the above criteria, are excluded from the invention.

The invention also relates to a compound as described for medicinal use. More specific, the compound may be used in the prophylaxis and/or treatment of overweight and/or obesity.

The details and particulars described above relating to the use of a compound according to the invention apply mutatis mutandis to the compounds according to the invention.

The invention also relates to a method of prophylaxis and/or treatment of overweight and/or obesity, the method comprising administering a pharmaceutical composition comprising a compound according to the invention by an intracerebroventricular route to a subject in the need thereof.

In another method according to the invention the method comprising administering a compound capable of crossing the blood bran barrier to a subject in the need thereof.

The invention further relates to a cosmetic method for reducing body weight, the method comprising administering a composition comprising a compound according to the invention.

An antagonist to GIP will most likely have an effect in increasing the body weight in a subject to be treated. Thus, the invention relates to use of an antagonist to GIP for the manufacture of a pharmaceutical composition for the prophylaxis and/or treatment of conditions caused or characterized by abnormally low body weight. As described above, the antagonist to GIP may be an antibody.

The invention also relates to the use of an antagonist to the GIP receptor for the manufacture of a pharmaceutical composition for the prophylaxis and/or treatment of conditions caused or characterized by abnormally low body weight.

The conditions to be prevented or treated by the administration of an antagonist to GIP or the GIP receptor may be selected from anorexia, cachexia, AIDS- or cancer-related wasting, and failure to thrive syndrom in newborn and young children.

Other aspects of the invention

As described above, the present inventors are the first to detect the presence of GIP in the brain of a mammal, by using a special antigen retrieval method to detect GIP with immunolabeling and by using very efficient primers for GIP.

Accordingly, the invention relates to a method for detecting an abnormal level of GIP in the brain of a mammal. The method may be used for diagnosis, disease monitoring and/or therapeutic monitoring of a disease characterized by an abnormal amount of GIP in the brain.

In one method according to the invention the disease may be characterized by that the level of GIP in the brain of a subject is low compared to a healthy subject. Examples of diseases characterized by a low level of GIP in the brain may be depression, mood disorders and eating disorders. Other examples of conditions that may be characterized by a low level of GIP in the brain are memory and learning disorders as well as dementia

The invention also relates to a method wherein the level of GIP in the brain of a subject is high compared to a healthy subject.

The invention also relates to a compound having SEQ ID NO 2 or analogues, functional analogues or fragments thereof as described herein for the manufacture of a pharmaceutical composition for prophylaxis and/or treatment of depression and/or mood disorders.

The invention further relates to a compound according to the invention for the manufacture of a pharmaceutical composition for prophylaxis and/or treatment of mania and manic/depressive disorders.

The invention also relates to use of a compound according to the invention for the manufacture of a pharmaceutical composition for prophylaxis and/or treatment of memory and/or learning disorders.

Furthermore, the invention relates to a pharmaceutical composition comprising a compound according to the invention together with one or more pharmaceutically acceptable excipients.

Other aspects of the invention appear from the appended claims. The details and particulars described above and in the claims and relating to the use of a compound according to the invention apply mutatis mutandis to the other aspects of the invention.

Administration

For medical use, the amount required of a compound according to the invention to achieve a therapeutic effect will vary according to the particular compound administered, the route of administration, the animal under treatment, and the particular disorder or disease concerned. A suitable systemic dose of a compound according to the invention for an animal suffering from, or likely to suffer from, any condition as described herein is typically in the range of about 0.1 to about 100 mg per kilogram of body weight, preferably from about 1 to about 10 mg/kg of animal body weight. It is understood that the ordinarily skilled physician or veterinarian will readily be able to determine and prescribe the amount of the compound effective for the desired prophylactic or therapeutic treatment.

In so proceeding, the physician or veterinarian may employ an intravenous bolus followed by an intravenous infusion and repeated administrations, as considered appropriate. In the methods of the present invention, the compounds may be administered, for example, orally, parenterally, by inhalation spray, topically, rectally, nasally, buccally, sublingually, vaginally, intraventricularly, or via an implanted reservoir in dosage formulations containing conventional non-toxic pharmaceutically-acceptable carriers, adjuvants and vehicles.

Parenteral includes, but is not limited to, the following examples of administration: intravenous, subcutaneous, intramuscular, intraspinal, intraosseous, intraperitoneal, intrathecal, intraventricular, intrasternal or intracranial injection and infusion techniques, such as by subdural pump. Invasive techniques are preferred, particularly direct administration to damaged neuronal tissue. While it is possible for the compound according to the invention to be administered alone, it is preferable to provide it as a part of a pharmaceutical formulation.

As mentioned above, to be effective therapeutically as central nervous system targets, the compounds used in the methods of the present invention should readily penetrate the blood-brain barrier when peripherally administered. An intraventricular route can still effectively administer compounds, which cannot penetrate the blood-brain barrier.

The compounds used in the methods of the present invention may be administered by a single dose or by multiple discrete doses.

For the methods of the present invention, any effective administration regimen regulating the timing and sequence of doses may be used. Doses of the compounds preferably include pharmaceutical dosage units comprising an efficacious quantity of active compound. By an efficacious quantity is meant a quantity sufficient to inhibit induce proliferation of cells and/or derive the desired beneficial effects therefrom through administration of one or more of the pharmaceutical dosage units. In one embodiment, the dose is sufficient to prevent or reduce the effects of neurodegenerative diseases.

An exemplary daily dosage unit for a vertebrate host comprises an amount of from about 0.001 mg/kg to about 50 mg/kg. Typically, dosage levels on the order of about 0.1 mg to about 10,000 mg of the active ingredient compound are useful in the treatment of the above conditions, such as levels being about 0.1 mg to about 1,000 mg. The specific dose level for any particular patient will vary depending upon a variety of factors, including the activity of the specific compound employed; the age, body weight, general health, sex, and diet of the patient; the time of administration; the rate of excretion; any combination of the compound with other drugs; the severity of the particular disease being treated; and the form and route of administration. Typically, in vitro dosage-effect results provide useful guidance on the proper doses for patient administration. Studies in animal models can also be helpful. The considerations for determining the proper dose levels are well-known in the art.

In methods of treating nervous insult (particularly acute ischemic stroke and global ischemia caused by drowning or head trauma), the compounds of the invention can be co-administered with one or more other therapeutic agents, such as agents which can reduce the risk of stroke (such as aspirin) and agents which can reduce the risk of a second ischemic event (such as ticlopidine).

To kill cells, inhibit cell growth, inhibit metastasis, decrease tumor or tissue size and otherwise reverse or reduce the malignant phenotype of tumor cells, using the methods and compositions of the present invention, one would generally contact a hyperproliferative cell with the therapeutic expression construct. The routes of administration will vary, naturally, with the location and nature of the lesion, and include, e.g. intradermal, transdermal, parenteral, intravenous, intramuscular, intranasal, subcutaneous, percutaneous, intratracheal, intraperitoneal, intratumoral, perfusion, lavage, direct injection, and oral administration and formulation.

Intratumoral injection, or injection into the tumor vasculature is specifically contemplated for discrete, solid, accessible tumors. Local, regional or systemic administration also may be appropriate. For tumors of (4 cm, the volume to be administered will be about 4-10 ml (preferably 10 ml), while for tumors of (4 cm, a volume of about 1-3 ml will be used (preferably 3 ml). Multiple injections delivered as single dose comprise about 0.1 to about 0.5 ml volumes. The compound according to the invention may advantageously be contacted by administering multiple injections to the tumor, spaced at approximately 1-cm intervals.

In the case of surgical intervention, the present invention may be used preoperatively, to render an inoperable tumor subject to resection. Alternatively, the present invention may be used at the time of surgery, and/or thereafter, to treat residual or metastatic disease. The perfusion may be continued post-resection, for example, by leaving a catheter implanted at the site of the surgery. Periodic post-surgical treatment is also envisioned.

Continuous administration also may be applied where appropriate, for example, where a tumor is excised and the tumor bed is treated to eliminate residual, microscopic disease. Delivery via syringe or catherization is preferred. Such continuous perfusion may take place for a period from about 1-2 hours, to about 2-6 hours, to about 6-12 hours, to about 12-24 hours, to about 1-2 days, to about 1-2 wk or longer following the initiation of treatment. Generally, the dose of the therapeutic composition via continuous perfusion will be equivalent to that given by a single or multiple injections, adjusted over a period of time during which the perfusion occurs. It is further contemplated that limb perfusion may be used to administer therapeutic compositions of the present invention, particularly in the treatment of melanomas and sarcomas.

Treatment regimens may very as well, and often depend on tumor type, tumor location, disease progression, and health and age of the patient. Obviously, certain types of tumor will require more aggressive treatment, while at the same time, certain patients cannot tolerate more taxing protocols. The clinician will be best suited to make such decisions based on the known efficacy and toxicity (if any) of the therapeutic formulations.

In certain embodiments, the tumor being treated may not, at least initially, be resectable. Treatments with therapeutic viral constructs may increase the resectability of the tumor due to shrinkage at the margins or by elimination of certain particularly invasive portions. Following treatments, resection may be possible. Additional treatments subsequent to resection will serve to eliminate microscopic residual disease at the tumor site.

A typical course of treatment, for a primary tumor or a post-excision tumor bed, will involve multiple doses. Typical primary tumor treatment involves a 6-dose application over a two-week period. The two-week regimen may be repeated one, two, three, four, five, six or more times. During a course of treatment, the need to complete the planned dosings may be re-evaluated.

The treatments may include various “unit doses.” Unit dose is defined as containing a predetermined-quantity of the therapeutic composition. The quantity to be administered, and the particular route and formulation, are within the skill of those in the clinical arts. A unit dose need not be administered as a single injection but may comprise continuous infusion over a set period of time. Unit dose of the present invention may conveniently be described in terms of mg/kg body weight.

For treating diseases such as psoriasis, the compound according to the invention is preferably administered as a lotion, cream, or any other composition suitable for administering a medicament on skin.

The compositions may be formulated according to conventional pharmaceutical practice, see, e.g., "Remington: The science and practice of pharmacy" 20^{th} ed. Mack Publishing, Easton PA, 2000 ISBN 0-912734-04-3 and "Encyclopedia of Pharmaceutical Technology", edited by Swarbrick, J. & J. C. Boylan, Marcel Dekker, Inc., New York, 1988 ISBN 0-8247-2800-9.

The choice of pharmaceutically acceptable excipients in a composition for use according to the invention and the optimum concentration thereof cannot generally be predicted and must be determined on the basis of an experimental determination thereof. Also whether a pharmaceutically acceptable excipient is suitable for use in a pharmaceutical composition is generally dependent on which kind of dosage form is chosen. However, a person skilled in the art of pharmaceutical formulation can find guidance in e.g., "Remington: The science and practice of pharmacy" 20^{th} ed. Mack Publishing, Easton PA, 2000 ISBN 0-912734-04-3.

### FIGURE LEGENDS

Figure 1 shows the expression of GIP mRNA in adult rat hippocampus; (a) Schematic picture of the microarray strategy. Arrows indicate the gene dots representing GIP mRNA on arrays from the three groups and the corresponding gene dots are enlarged at the bottom right of the panel; (b) PCR analysis of the GIP gene in hippocampus (lane 2) shows a band corresponding to 220bp, as do the positive control in mRNA from the small intestine (lane 4). Expression of RPL27A was used as an internal standard. Negative controls are without cDNA (lane 1) or hippocampal mRNA without RT (lane 3); (c) In situ hybridization with probes for GIP mRNA shows a weak but specific localization in the granule cell layer of the dentate gyrus as well as the CA1-CA3 region; (d) Semiquantitative PCR comparisons of hippocampal mRNA from SHR-males, SD-males and SD-females shows the same expression pattern as the microarrays. When using only 30 cycles the band for GIP could not be detected in SD-females.

Figure 2 illustrates the presence of GIP immunoreactivity granule cell neurons of the adult hippocampus; (a) shows that GIP immunoreactivity was found as a cytoplasmatic staining in the granule cell neurons; (b) demonstration of co-localization of GIP-immunoreactivity (green) and Calbindin (red) in the hippocampal granule cell layer; (c) demonstration of co-localization of GIP (red) and NeuN (green); (d) shows that the subgranular layer contains progenitor cells, here detected by BrdU-immunoreactivity (green) and granule cells are stained for Calbindin (red). The inner part of the granule cell layer facing the hilus area was only GIP positive and not Calbindin positive; (e) comparison of levels of immunoreactivity in brain slices from SHR-males, SD-males and SD-females; (f) immunoreactivity in brain slice from SHR-males; (g) immunoreactivity in brain slice from SD-males; (h) immunoreactivity in brain slice from SD-females. Scale bar = 50 µm in a-d and 100 µm in f-h.

Figure 3 shows that the GIP receptor is present in both progenitor cells and mature neurons in adult hippocampal tissue; (a) PCR analysis of the GIP receptor gene revealed a band corresponding to 540bp in mRNA isolated from cultured adult hippocampal progenitor cells. Lane 1 (+) shows RNA from cells that have been cultured with FGF-2, thereby kept in an undifferentiated state. Cells were also allowed to differentiate for 4 days, 6 days, 10 days and 14 days (lane 2 – 5) through withdrawal of FGF-2. RNA from hippocampus was used as a positive control (lane H) and mRNA from spleen (lane S) as a negative control. RPL27A was used as an internal standard; (b) In situ hybridization with probes for the GIP receptor showed weak but specific expression in the dentate gyrus, especially in the granule cell layer; (c) Cultured adult hippocampal progenitor cells exhibited immunoreactivity for the GIP-receptor; (d) shows that the GIP receptor (red) was found in cells expressing Nestin (green), that marks undifferentiated neuronal cells; (e) shows that the GIP receptor (red) was also observed in cells expressing more mature markers as Map2ab (green); (f) shows Western blot of both hippocampal protein (H) and protein from adult hippocampal progenitor cells (P). A band corresponding to 70 kDa, the size of the GIP-receptor is seen; (g) The granule cell layer, marked with NeuN (green); (h) The granule cell layer also contains cells that also express the GIP-receptor (red); (i) shows a merged picture of co-localisation of NeuN and the GIP-receptor; (j) shows that newly born cells in the granule cell layer also express the GIP-receptor, here shown through co-localisation of BrdU (green) and the GIP-receptor (red) (j). Scale bar = 10 µm in (j), otherwise 25 µm.

Figure 4 shows that GIP induces proliferation of adult hippocampal progenitor cells both in culture and in vivo; (a) shows the amount of DNA in cultured adult hippocampal progenitor cells following incubation with different concentrations of GIP for 48 h; (b) shows the amount of DNA in cells treated with a combination of FGF-2 (20ng / ml) and GIP (1 nM). Cells were cultured without FGF-2 as a negative control and given a basal level of 100 %. DNA content is calculated as a percentage of content obtained from cells grown without FGF-2. Values are means ± SEM of eight (GIP and GIP + FGF-2) or four (FGF-2) different experiments (each experiment represents the mean of four different culture wells). * P < 0.05; ** P< 0.01; *** P < 0.001 (one-way ANOVA followed by Fisher’s post hoc test); (c) shows brain sections stained immunohistochemically for BrdU in rats that had been given i.c.v. PBS; (d) shows brain sections stained immunohistochemically for BrdU in rats that had been given i.c.v. GIP; (e) shows the density of BrdU-positive cells (cells per cubic millimeter of sample volume) in the granule cell layer determined stereologically. The quantification of BrdU-positive cells in the adult rat hippocampus showed that GIP-treated animals (n = 5) exhibited 86% more BrdU-immunoreactive cells than animals treated with PBS (0.1 M; n = 6). Means ± SEM are given * p < 0.05 (one-way ANOVA followed by Fisher’s post hoc); (f) is an illustration of that both mature cells as well as progenitor cells express the GIP receptor, but GIP itself is produced by mature granule cells in the whole GCL. Progenitor cells are located in an environment where GIP is produced and respond to the peptide by an increase in cell proliferation. Scale bar = 100 µm.

Figure 5shows the effect of GIP on weight gain in rats.

The following example is intended to illustrate the invention without limiting it thereto.

### EXAMPLES

Animals: All experimental protocols were approved by the Animal Ethics Committee of Göteborg University. Animals used for the cDNA array were obtained from Möllegaard Breeding Center (Ejby, Denmark), but for all other experiments they were obtained from B&K Universal (Stockholm, Sweden). In the study was used male Spontaneously Hypertensive rats (SHR) that exhibit a significantly higher cell proliferation and net neurogenesis in the hippocampal dentate gyrus (DG) than male Sprague-Dawley rats (SD) ¹¹.

Example 1

Expression of the GIP gene in hippocampus co-varies with cell proliferation rates in rat DG

To investigate genes that might be associated with neuronal proliferation in the young adult rat hippocampus, RNA were isolated from three groups of rats known to differ with regards to neural progenitor cell proliferation in the adult DG

Materials and methods

Atlas cDNA Array: Male SHR (n = 5), and male (n = 5) and female (n = 5) SD rats were sacrificed at five weeks. Hippocampus from one half of the brain was used for RNA isolation and the other half of the brain for immunofluorescence. Total RNA from each hippocampus was separately prepared according to the Atlas™ Pure Total RNA Labeling System User Manual (PT3231-1, Cat #: K1038-1) and pooled. Preparations of cDNA probes, hybridization to arrays and development of X-ray films were made according to the Atlas™ cDNA Expression Arrays User Manual (PT3140-1). Array experiments were performed twice on separate sets of rats. Data analyses were performed using the software AtlasImage™ (Clontech) according to AtlasImage™ 1.5 User Manual

RT-PCR: Total RNA was isolated from cultured adult hippocampal progenitor cells, rat hippocampus, small intestine and spleen⁴³. All reagents were obtained from Promega, Madison, WI and the cDNA was cycled using a thermal cycler (Perkin Elmer 2400) for 35 cycles. PCR primers for GIP were designed by Clontech (GIP-P1, AAG AGG TTG AGT TCC GAT CCC ATG C; GIP-P2, GAT TGT CCT GCC AGC TCC AAA GCC) and the primers for the GIP receptor have been previously described¹⁵. As an internal standard, PCR primers detecting ribosomal protein 27A were used.

Sequencing: Sequencing was performed on PCR products using ABI Prism BigDye Terminator Cycle Sequencing Ready Kit (Applied Biosystem) and the same primers as used for RT-PCR. The products were precipitated with 95% ethanol and 3 M NaAc and resuspended in Template Suppression Reagent (Applied Biosystem) and further analysed on ABI PRISM 3100 Genetic Analyzer.

Results

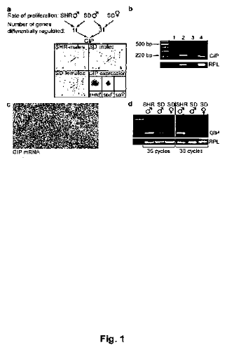

Hippocampal RNA was isolated from normal prepubescent male SHR and male and female SD rats as summarized in Figure 1a and used to synthesize cDNA probes for hybridization to an ATLAS rat 1.2 cDNA Array. The reason for approaching this question with a cDNA strategy was to perform a simple screening and to identify a novel hypothesis to continue investigating with other methods. The purpose of this analysis was not to characterize all the differences in hippocampal gene regulation in the different groups of rats. The hybridization results are shown in Figure 1a, where a dark spot represents each gene in the Array. A comparison of hippocampal gene expression profiles from male SHR to male SD rats (SHRs have a higher rate of progenitor cell proliferation when compared with SD rats) revealed 11 differentially expressed genes (with more than 4 fold difference in expression) between the two groups. Subsequently there was performed a second comparison between male and female SD rats (males have a higher rate of progenitor cell proliferation). The results revealed 31 differentially expressed genes. Data was compiled from the two comparisons, and it was attempted to identify genes that demonstrated an expression profile that co-varied with the in vivo proliferation level of cells in DG in both comparisons. GIP was up-regulated in male SHR compared to SD males, and in SD males compared to SD females. GIP was the only gene of 1200 genes analyzed that exhibited this pattern.

Expression of the GIP gene in brain tissue was confirmed using RT-PCR. RNA from rat hippocampus and rat small intestine (positive control) was reverse transcribed and aliquots of the same cDNA used for all reactions. RPL 27A RNA was used as an internal standard as seen in Figure 1b. In RNA from both hippocampus and small intestine a band corresponding to 220 bp was observed and subsequently sequenced. Sequencing of the PCR product confirmed expression of the GIP gene in the rat hippocampus. Members of the VIP/secretin/glucagon family have a similar amino acid sequence near the N-terminal part of the cDNA that encodes the active peptide, but are otherwise very different ^{24,25}. Our reverse GIP RT-PCR primer hybridized to the C-terminal extension, which exhibits considerable amino acid and cDNA sequence divergence. To verify expression of GIP mRNA in rat hippocampus we also performed in situ hybridization of brain sections using two different oligonucleotide probes. GIP mRNA expression was demonstrated, although weak, in the CA1-CA3 region and the DG including the granule cell layer ( see Figure 1c). GIP expression was also higher in RNA from male SHR compared to male SD rats and RNA from female SD rats had the lowest expression when analyzed using semi-quantitative RT-PCR (see Figure 1d). The 220 bp band could not be detected in female SD rats when only cycling 30 times.

Example 2

Expression of the GIP peptide in hippocampus

The example shows the presence of GIP in hippocampus of adult rats as determined by immunohistochemical methods

Methods

Immunofluorescence staining: Cell cultures: Clonal adult hippocampal progenitor cells from rat ⁷ were cultured as previously described⁴⁴. Primary antibodies; rabbit GIP receptor (1:500) ⁴⁵ and mouse Nestin (1:500, PharMingen, Becton Dickson, Franklin Lakes, NJ). Rat brains: Sectioning, staining and detection of immunofluorescence was performed as previously described⁴⁶. Primary antibodies: monoclonal mouse GIP (3.65H; 1:1000, kindly provided by Dr. Alison Buchan, UBC, Canada), polyclonal rabbit GIP (1:100, Chemicon), rabbit GIP receptor (1:500), mouse BrdU (1:400, Boeringer Mannheim), rabbit GFAP (1:500, Dako, Glostrup, Denmark), rabbit Calbindin D_{28K} (1:500, Swant, Bellinzona, Switzerland), mouse NeuN (1:30, Chemicon). Secondary antibodies for both cultured cells and brain sections were Alexa Fluor 488 conjugated anti-mouse and Alexa Fluor 594 conjugated anti-rabbit (both 1:400, Molecular Probes, Leiden, Netherlands). For antigen retrieval of GIP, sections were microwaved for 4 x 2min (Moulinex Micro-Chef MO55; 650W/230V/50Hz) in TBS.

Immunoquantification. Brain sections including hippocampus of male SHR and male and female SD rats were stained using a monoclonal GIP antibody (se above). Sections were anatomically compared so that the same equivalent locations were chosen. Two sections per rat and four rats per group were stained. Quantification was carried out using a computer program from Nikon-Mikael.

Results

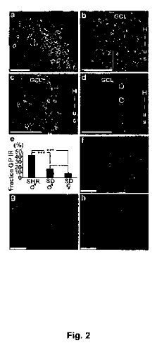

The presence of GIP immunoreactivity in the adult rat hippocampus was examined immunohistochemically using a monoclonal antibody against GIP as well as a polyclonal. As seen in Figure 2a, the granule cell layer in hippocampus contained a large amount of GIP immunoreactivity with a characteristic cytoplasmic staining pattern. No co-labeling of GIP with the glial cell marker GFAP was observed (not shown) but cells in the hippocampal granule cell layer showed co-localization of GIP immunoreactivity with the neuronal marker Calbindin and NeuN (see Figure 2b-c). Thus, GIP immunoreactivity is expressed throughout the DG, including the inner subgranular cell layer, an area of active proliferation and neurogenesis in the adult mammal ^{1,4,10}. The close proximity of the progenitors to cells producing GIP indicates that they are probably exposed to the peptide. We were not able to perform any co-labeling with GIP and BrdU as the DNA denaturation step required for BrdU-labeling resulted in loss of GIP immunoreactivity. However, although we were not able to perform co-labelling with GIP and BrdU, staining for BrdU and Calbindin shows the location of newly formed cells in the subgranular cell layer, indicating the close proximity of BrdU- and GIP-labeled cells (see Figure 2d). GIP immunoreactivity was found in the DG of all groups, that is, male SHR as well as female and male SD rats.There was also a corresponding significant difference in immunoreactivity levels for GIP in the hippocampal granule cell layer of these three groups, confirming the variation observed on the cDNA array also on protein level (see Figure 2 e-h).

Indeed, reports in the past have been conflicting concerning the presence of GIP immunoreactivity in mammalian pancreas due to the usage of different antibodies ^{26,27}. The specificities and sensitivities of different GIP antibodies have been investigated and conclusions drawn that a monoclonal C-terminal-specific antibody is the most suitable ^{26,27}. To conclude that we have not detected an other member of the VIP/secretin/glucagon family of peptides, the antibody used in this study is monoclonal and C-terminal-specific and has also been tested for its specificity through preincubation with VIP, secretin, glucagon and somatostatin ^{26,28}. Therefore we reasonably conclude that the adult rat brain produces GIP.

Example 3

Expression of the GIP receptor in adult hippocampal progenitor cells

The example shows that hippocampal progenitor cells express the GIP receptor, and that cells in the neurogenic region of the brain produce GIP under physiological conditions.

Methods

In situ hybridization. Male Sprague-Dawley rats were decapitated and the brains were sectioned at 14 µm thickness in a cryostat (Dittes, Heidelberg, Germany) and thaw-mounted onto pretreated glass slides (ProbeOn™, Fisher Scientific, Pittsburgh, PA, USA). Using MacVector™ software (IBI, New Haven, CT, USA) oligonucleotide probes were selected based on optimum ratio of guanosine + cytosine/total nucleotide numbers (50-65%) and minimal homology (not greater than 80%) with GenBank-entered sequences. Oligonucleotide probes were made reversed and complementary to GGCTTTGGAGCTGGCAGGACAATCT CAGAGAAACGAGGAGAAAGAGGC (nucleotides 313-360) and TGCTGGCCCCC GACCACGAGGCCCAAGGTATGCAGAGGGGACTTTCAT (nucleotides 148-195) of rat GIP mRNA^{16,17} and GTACAGGTGAGCACTGACTTGGGCTGAAGCTCAAGAGTTG GTTCTGCC (nucleotides 61-108) and CCTGTTCACGTCTTTCATGCTGCGAGCAGGGG CCATCCTCACCCGAGA (nucleotides 682-729) of rat GIP-R mRNA¹⁵ and synthesized (MWG Biotech, Ebersberg, Germany). The probes were labeled with ³³P-dATP (NEN, Boston, MA, USA) at the 3'-end using terminal deoxynucleotidyltransferase (Amersham Ltd., Amersham, UK) and purified using ProbeQuant G-50 Micro Columns (Amersham Pharmacia Biotech, Inc., Piscataway, NJ, USA). The specific activity of the labeled probes were 3 x 10⁹ cpm/(g. In situhybridization was carried out essentially as described⁴⁷. Tissue sections were air-dried and incubated with a hybridization solution containing 0.5 ng of labeled probe/slide. The hybridization solution contained 50% deionized formamide (J.T. Baker Chemicals, Deventer, The Netherlands), 4 x SSC (1 x SSC = 0.15 M sodium chloride, 0.015 M sodium citrate), 1 x Denhardt's solution [0.02% bovine serum albumin, 0.02% Ficoll (Pharmacia, Uppsala, Sweden), 0.02% polyvinylpyrrolidone], 1% N-lauroylsarcosine, 0.02 M NaPO4 (pH 7.0), 10% dextran sulphate (Pharmacia), 500 µg/ml denatured salmon testis DNA (Sigma, St. Louis, MO, USA) and 200 mM dithiothreitol (LKB, Stockholm, Sweden). After 16 hours of incubation, the slides were rinsed in 1 x SSC for 4 times 15 minutes at 56°C and allowed to cool down to room temperature, washed in distilled water, transferred rapidly through 60% and 95% ethanol. The ³³P-dATP–labeled sections were apposed to (-max autoradiography film (Amersham). The films were exposed for two months and developed with Kodak LX 24 and fixed with Kodak AL4. Autoradiography films were scanned using a UMAX PowerLook 3000 scanner (Umax Technologies, Inc., Dallas, Texas, USA) and processed using Adobe Photoshop 5.5 software (Adobe, Inc., San Jose, CA, USA).

Western blot. Adult hippocampal progenitor cells were plated on polyornithine (PORN)/laminin coated plates at a density of 2 x 10⁴ cells per cm². The cells were lysed in cold RIPA buffer containing 1 % protease inhibitor cocktail (Sigma) and centrifuged at 12,000 x g 5min at 4(C. The supernatant was analyzed for protein concentration using the Lowry assay⁴⁸. The Western blot was performed using polyacrylamide gels (with 10% separation gel, pH 8.8, and 4% stacking gel, pH 6.8, in 0.1% SDS) run for 2 to 3 h at 20 mA using a Protean Cell apparatus (Biorad, CA, USA). Samples of 15 µg protein and a negative control using rat serum was loaded to the gel. Protein was transferred to a PVDF membrane (Immobilon-P, Millipore, Bedford, MA) at 80 mA overnight. The membranes were washed in PBS, blocked in 5% milk protein for 1 h and then incubated with primary rabbit GIP receptor (1:500) antibody diluted in 5% milk protein over night. Controls without primary antibody was also performed. After washes in PBS-T the membranes were incubated in secondary antibody; HRP-conjugated donkey anti-rabbit (1:1000; Amersham). After washes in PBS-T, the membranes were treated with chemiluminescense substrate (Boehringer-Mannheim GmBH) and recorded on film.

Results

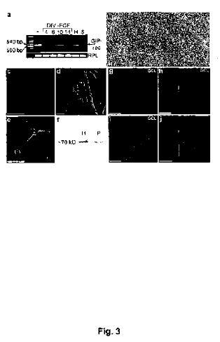

Cultured adult hippocampal progenitor cells were analyzed for the presence of the GIP receptor to investigate whether these cells have the ability to respond to GIP. RT-PCR results revealed expression of the GIP receptor gene in these cells (see Figure 3a). Expression of the GIP receptor gene in hippocampal tissue was also observed in accordance with others¹⁵. Expression of the GIP receptor gene was highest in cells cultured with fibroblast growth factor-2 (FGF-2). FGF-2 is a well-documented proliferative agent of these cells. Expression of the GIP receptor gene decreased following removal of FGF-2, which allows the cells to differentiate. GIP receptor mRNA expression was also investigated through in situ hybridization of brain sections using two different oligonucleotide probes, which confirmed, although weak, the expression in the hippocampal granule cell layer (see Figure 3b). To investigate the presence of the GIP receptor in cultured adult hippocampal progenitor cells, immunocytological staining on these cells using a GIP receptor antibody was performed as shown in Figure 3c. Co-localization of the GIP receptor and Nestin, a marker of neuronal progenitor cells, demonstrate that the receptor is present in undifferentiated progenitor cells as shown in Figure 3d, but co-localization with more mature neuronal markers, such as Calbindin, Map-2ab and beta-Tubulin was also observed (see Figure 3e). This indicates that the GIP receptor not only is confined to undifferentiated cells. Presence of the GIP receptor in cultured progenitor cells was also found using Western blot where a band of around 70 kD was observed as shown in Figure 3f. Immunohistochemical detection of the GIP receptor in brain sections revealed expression in the whole hippocampal granule cell layer (see Figure 3g-i) as well as co-localization with BrdU, again demonstrating that the GIP receptor not only is located in immature cells (see Figure 3j). Thus, cells in neurogenic regions of the brain produce GIP under physiological conditions and hippocampal progenitor cells express the GIP receptor, suggesting that GIP may influence aspects of progenitor cell proliferation.

Example 4

GIP increases proliferation in cultured adult hippocampal progenitor cells

The example demonstrate that hippocampal GIP gene expression is up-regulated in association with increased levels of progenitor cell proliferation and that GIP is present in the DG in the vicinity of progenitor cells

Methods

Proliferation assay. Hippocampal progenitor cells were seeded at 0.2 x 10⁴ cells/cm² on 24-well plates in culture medium containing human FGF-2 (20ng/ml) and left to grow for 48 h. After a further 48 h of growth without FGF-2, cells were incubated with porcine GIP at different concentrations (Sigma), FGF-2 alone or a combination of both GIP (1 nM) and FGF-2 for 48 h. Cell proliferation assay was performed using the CyQUANT Cell Proliferation Kit (Molecular Probes, Eugene, OR) and a GENios microplate reader (TECAN Austria GmbH, Grödig, Austria) according to the instructions of the manufacturer.

Thymidin-assay. Hippocampal progenitor cells were seeded at 0.5 x 10⁴ cells/cm² on 48-well plates in culture medium and left to grow for 48 h. The cells were then labeled with methyl-[³H]-thymidine and incubated with GIP (1 nM) or FGF-2 (20ng/ml) for 24 h. Cells were lysed in 0.4 M NaOH, transferred to scint vials, mixed with 0.4 M HCl and assayed for DNA synthesis by scintillation spectrometry. The mean for each experiment was calculated from four different culture wells and each experiment was performed 12 times.

Results

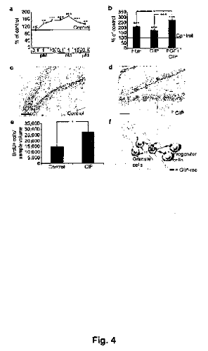

The results demonstrate that hippocampal GIP gene expression is up-regulated in association with increased levels of progenitor cell proliferation and that GIP is present in the DG in the vicinity of progenitor cells. Progenitor cells in turn express the GIP receptor and can therefore respond to the peptide. Subsequent studies were designed to investigate whether GIP might be involved in the regulation of proliferation of neuronal progenitor cells. This was achieved using a commercial proliferation assay. Culturedadult hippocampal progenitor cells were incubated with synthetic porcine GIP at different concentrations and compared to a control. GIP increased the rate of cell proliferation in a dose-dependent fashion with doses from 1 pM to 0.1 µM resulting in significant increases as shown in Figure 4a. The greatest effect was achieved at a GIP concentration of 1 nM (74.5 ± 14.4% increase relative to control; n = 8), but at 0.1 pM the proliferative effect was absent. Cells were also treated with FGF-2 (20 ng/ml) alone and with a combination of GIP (1 nM) and FGF-2 (20 ng/ml). FGF-2 alone resulted in a 112.3 ± 20% (n = 4) increase relative to control. At the beginning of the experiment, around 6 x 10³ cells were seeded in a well and at the end of the experiment there was around 1.5 x 10⁵ cells in a control well with out FGF, 3 x 10⁵ cells in a well with FGF and 2.5 x 10⁵ cells in a well with GIP. When incubating cells with GIP in addition to FGF-2, a synergistic effect on proliferation was observed, with an increase in cell growth of 171.8% ± 16.1% (n = 8) relative to control (see Figure 4b). These experiments show that GIP has slightly more than half of the proliferative effect of FGF-2. This result was also confirmed using a methyl-[³H]-thymidin incorporation assay, where 1 nM GIP increased thymidin incorporation with 32.4 ± 3.3 % (n = 12) and FGF-2 with 60.1 ± 7.1 % (n = 12) compared to control. Indeed, GIP acts on proliferation in cultured adult hippocampal progenitor cells.

Example 5

GIP does not influence the rate of cell death in cultured adult hippocampal progenitor cells

Methods

Apop-Tag. Hippocampal progenitor cells were seeded at 1 x 10⁴ cells/cm² on glass coverslips and treated the same way as for the proliferation assay and incubated with GIP (1 nM) or FGF-2 (20 ng/ml). Cells were fixed and stained for apoptosis according to the Apop Tag kit user manual (Apop Tag S160 direct, Intergene Company, Purchase, NY, USA). A negative control without TdT-enzyme was included and positive controls with addition of H₂O₂ (100 µM and 1 mM) and DNase I (1 µg/ml) was included. In the last washing step the cells were incubated with the nuclear dye bisBenzimide (Hoechst 33258, Sigma) for 30 min. Apoptotic or dead cells were identified by green fluorescence in the nuclei and Hoechst nuclear dye was used to discriminate total number of cells. Three coverslips per experiment was stained from four different experiments. Positive cells was quantified by scoring the immunoreactivity of 1000–3000 cells systematically observed in 6 nonoverlapping fields in each coverslip.

LDH-activity. Release of lactate dehydrogenase (LDH) from dying cells was measured using a routine photometric method (Dept. of Clinical Chemistry, Sahlgrenska University Hospital, Sweden). Cell culture medium was collected from culture wells seeded for the Apop Tag staining (see above). . The mean for each experiment was calculated from three different culture wells and each experiment was performed 4 times. The coefficient of variation for the assay was 1.7 % and the assay standard curve was linear for enzyme activities between 0.1–20 µkat/ dm³.

Results

To investigate if GIP rather than having a mitogenic effect might instead have a survival-effect, the ApopTag kit for detection of dead cells was used. Cultured adult hippocampal progenitor cells were treated the same way as for the proliferation experiments and fixated the last day. As positive controls was used DNaseI treatment (1 µg/ml) for 10 min after fixation or induction of cell death using 100 (M and 1 mM H₂O₂ for 30 min before fixation. This treatment induced cell death in most cells, as judged by immunoflouorescence. Cell death in the control experiments without FGF-2 (20ng/ml) or GIP (1nM) was 3.31 + 0.67% and not statistically different from cell death in cells treated with GIP. FGF-2 had a slightly decreasing effect on cell death with only 0.96 + 0.21%. In each experiment was also determined the extracellular level of released lactate dehydrogenase (LDH) from dying cells. Results revealed no statistical difference between controls and cells treated with GIP or FGF-2, verifying the assumption that GIP does not influence survival of these cells but most likely acts to stimulate proliferation.

Example 6

GIP increases proliferation in adult rat DG

This experiment shows that GIP has effects on proliferation of hippocampal progenitor cells in their natural environment.

Methods

Intracerebroventricular GIP infusion. Adult male SD rats (260-280 g; B&K Universal, Sweden) were intubated and ventilated with isoflurane in an O₂/N₂O mix (30:70). An infusion cannula connected to an osmotic pump (Alzet brain infusion kit II and Alzet 2001 osmotic pump, Alza Scientific Products, Palo Alto, CA) was placed in the 3rd cerebral ventricle (0.3 mm posterior from Bregma along the midline, 5 mm below skull surface). Each rat was infused (1 µl/hr) for 5 days with either GIP (1.92 nmol/day; n = 5) or vehicle (0.1 M PBS; n = 6) and sacrificed the last day. All animals received a single daily intraperitoneal injection of Bromodeoxyuridine (BrdU; 50 mg/kg of body weight; Boehringer Mannheim; Scandinavia AB, Bromma, Sweden).

Immunohistochemistry and cellcounting. Brains were sectioned and stained for BrdU using a primary mouse BrdU antibody (1:400, Boeringer Mannheim) and biotinylated horse anti-mouse IgG (1:125) secondary antibodies (Vector Laboratories, Burlingame, CA) as previously described¹¹. For each animal, the total number of BrdU-positive cells in the granule cell layer, including the subgranular layer, and their corresponding sample volume were determined in 12 immunoperoxidase-stained, 40-µm-thick coronal sections taken 240 µm apart. The cross sectional areas were obtained using a CCD camera linked to a digital imaging system (Nikon, Sweden). Results are expressed as BrdU-positive cells per sample volume.

Statistics. Comparisons between groups were made using one-way ANOVA followed by a Fisher’s post hoc test, when appropriate throughout the study. A p-value < 0.05 was considered statistically significant. All values are expressed as the means ± SEM.

Results

To confirm whether GIP increases proliferation of cells in the adult DG in vivo, the number of newly formed cells in the rat subgranule cell layer after chronic i.c.v. infusion of GIP was analyzed. Adult male SD rats were given GIP or vehicle infusions in combination with daily BrdU injections for 5 days to label dividing cells. The number of newly generated cells in the adult subgranule cell layer was determined by a stereological analysis of the number of BrdU positive cells in the DG (see Figure 4c-d). In animals that underwent GIP-treatment, the number of BrdU+ cells in the granule cell layer was 27969 ± 5795 cells/mm³ (n = 5) compared to 14986 ± 1831 cells/mm³ (n = 6) in PBS-treated animals, which corresponds to an 86% (p < 0.05) increase in GIP-treated animals as seen in Figure 4e. This experiment shows that GIP also has effects on proliferation of hippocampal progenitor cells in their natural environment. Both mature cells as well as progenitor cells express the GIP receptor, but GIP itself is produced by mature granule cells in the whole GCL as shown in Figure 4f. Progenitor cells are located in an environment where GIP is produced and respond to the peptide by an increase in cell proliferation. The effect of GIP on mature granule cells are yet to be investigated.

Discussion of results in Example 1 to 6

Although the regulation of proliferation and differentiation of neural progenitor cells during CNS development has been extensively studied ²⁹, the knowledge regarding the factors that influence adult neurogenesis is more limited. Investigations of the cues and stimuli that influence proliferation and recruitment of neuronal progenitor cells in the adult brain are important to further understand cellular diversity and possible pathological conditions coupled to neurogenesis. In the present inventionis not only describe the identification of GIP as a proliferative peptide, but also its presence in the mammalian brain for the first time.

In the present invention, expression of GIP in the hippocampal granule cell layer was observed, an area of active proliferation and neurogenesis in the adult mammal ^{1,4,10}. Furthermore, convincing evidence that GIP does indeed influence cell proliferation of hippocampal progenitor cells is provided. GIP was first detected when it showed an up-regulated hippocampal gene expression in groups of rats naturally exhibiting a higher rate of progenitor cell proliferation in the dentate gyrus. This was later confirmed both with semiquantitative PCR and with comparisons of levels of GIP immunoreactivity in the granule cell layer. Hippocampal progenitor cells was shown to express the GIP receptor gene and protein both in cultures and in vivo. In the adult rat hippocampus, progenitor cells are located close to cells producing GIP and are presumably exposed to the peptide (Figure 4f). The hypothesis that GIP might influence progenitor cell proliferation was confirmed in cultures following administration of synthetic GIP. GIP increased the proliferation rate of cultured adult hippocampal progenitor cells in a dose-dependent manner. It was also found that expression of the GIP receptor RNA in cultured cells was higher in undifferentiated progenitor cells than in cells allowed to mature into more differentiated forms, suggesting that the receptor is down-regulated as the cells differentiate, again pointing to a role for GIP in stimulating proliferation. Interestingly, GIP and FGF-2 acted synergistically with regards to proliferation of cultured progenitor cells. This is probably explained by the up-regulation of GIP receptor RNA induced by FGF-2 thereby increasing the cells responsiveness towards GIP, similar to the effect of FGF-2 on the insulin-like growth factor-I receptor³⁰. Furthermore, i.c.v. infusion of GIP into adult rats resulted in a significant increase in proliferation of cells in the granule cell layer of the hippocampus as detected by BrdU incorporation, thereby demonstrating that GIP influences proliferation of these cells in vivo as well as in culture.

The results are consistent with the ability of GIP to induce proliferation as demonstrated by an increase in [3H] - thymidine incorporation in quiescent adrenal tumor cells ³¹ as well as acting as a growth factor for β (INS-1) cells³². This might point in the direction for GIP acting mitogenic also in celltypes of non-neural origin. Indeed other members of this family of neuropeptides have growth stimulating qualities ²⁴. PACAP reportedly increases proliferation in cultured granule cells from developing cerebellum ³³ and in sympathetic neuroblasts ^{34,35}, but has also been demonstrated to inhibit precursor mitosis in the developing cerebral cortex³⁶. Moreover, VIP acts as a potent mitogen during embryonic brain development ^{37, 38}, GHRH stimulates somatotroph cell proliferation ^{39,40} and GLP-2 stimulates cell proliferation in the intestine ^{41,42}. Many of the peripheral effects of GIP can be viewed as anabolic processes^{21,22}, and this could also be the case for neuronal tissue, where production and secretion of GIP might be a signal to start to maintain neuronal components, including synthesis of new cells in exchange for lost ones, thereby contributing to a ongoing turnover of neuronal cells in the brain.

Although expression of the other members of the secretin-glucagon family of gastrointestinal regulatory polypeptides has been described in the brain ²⁴, previous efforts to detect GIP mRNA in the brain have been unsuccessful ^{15,16}. The reasons for the failure of detecting GIP previously are unknown, but the present study provided conclusive evidence for its presence.

The current study describes, for the first time, the presence of GIP expression and GIP immunoreactivity in the adult rat brain. GIP is the last of the group of secretin-glucagon family of gastrointestinal peptides to be discovered in the brain. Moreover, it demonstrates that GIP influences hippocampal progenitor cell proliferation and therefore may be an important regulatory molecule for neural progenitor cell proliferation in the adult mammalian brain. This finding calls for investigations of whether GIP also may act as a potential anabolic and growth-stimulating factor for cell types of different origins.

Example 7

GIP’s effect on memory/learning

Methods

Adult male Sprague-Dawley rats (260-280 g; B&K Universal, Sweden) were intubated and ventilated with isoflurane in an O₂/N₂O mix (30:70). An infusion cannula connected to an osmotic pump (Alzet brain infusion kit II and Alzet 2001 osmotic pump, Alza Scientific Products, Palo Alto, CA) was placed in the 3rd cerebral ventricle (0.3 mm posterior from Bregma along the midline, 5 mm below skull surface). Each rat was infused (1 µl/hr) for a week with either GIP (1.92 nmol/day; n = 15) or vehicle (0.1 M PBS; n = 15) and also received a single daily intraperitoneal injection of Bromodeoxyuridine the five first days (BrdU; 50 mg/kg of body weight; Boehringer Mannheim; Scandinavia AB, Bromma, Sweden). The rats were then anaesthetized, the pumps were removed and the rats were allowed to recover for twenty days. The rats were then tested in the Morriz water maze with a video-tracking system for four consecutive days. The time to reach the platform (latency) and the length of the swimming path were monitored. The escape platform was hidden 1 cm below the surface of the water at a fixed position. The water was made opaque by adding dry milk powder and was kept constant at 22°C throughout the test. Each rat was tested in four trials each day. A trial consisted of placing a rat by hand into the water at one of four starting locations equally spaced around the pool’s perimeter. A block of four trials included one trial from each of the starting locations. Each trial lasted 45 s. Rats that failed to find the hidden platform within 45 s were designated as having a 45 s latency and were put on the platform and allowed to stay there for 15 s.

Example 8

GIP’s effect on weight gain

Methods

Rats (male Sprague-Dawley) were given either GIP (6 rats, 1.92 nmol/day) or phosphate buffered saline (PBS) as control-solution (7 rats), intraventricularly in the brain by osmotic mini-pumps. The rats were given substance during five days, and then sacrificed.

The weight of each rat was recorded and the total weight gain during the five days calculated.

Results

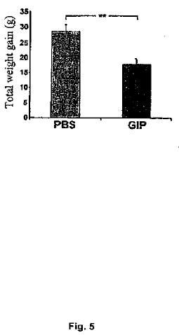

The rats normally show a weight gain of about 5g/day. The rats that were given PBS gained in average 28.5g during the five days, while the rats that were given GIP only gained 17.9g, i.e. 63% of the weight gain seen in the PBS treated rats. The results are shown in Figure 5, showing lower weight gain in GIP-treated rats.

### REFERENCES

1. Altman, J. & Das, G.D. Post-natal origin of microneurones in the rat brain. Nature 207, 953-956 (1965).

2. Kaplan, M.S. & Hinds, J.W. Neurogenesis in the adult rat: electron microscopic analysis of light radioautographs. Science 197, 1092-1094 (1977).

3. Goldman, S.A. & Nottebohm, F. Neuronal production, migration, and differentiation in a vocal control nucleus of the adult female canary brain. Proc. Natl. Acad. Sci. U S A 80, 2390-2394 (1983).

4. McKay, R. Stem cells in the central nervous system. Science 276, 66-71 (1997).

5. Eriksson, P.S. et al. Neurogenesis in the adult human hippocampus. Nat. Med. 4, 1313-1317 (1998).

6. Kuhn, H.G., Dickinson-Anson, H. & Gage, F.H. Neurogenesis in the dentate gyrus of the adult rat: age-related decrease of neuronal progenitor proliferation. J. Neurosci. 16, 2027-2033 (1996).

7. Palmer, T.D., Takahashi, J. & Gage, F.H. The adult rat hippocampus contains primordial neural stem cells. Mol. Cell. Neurosci. 8, 389-404 (1997).

8. Åberg, M.A., Åberg, N.D., Hedbacker, H., Oscarsson, J. & Eriksson, P.S. Peripheral infusion of IGF-I selectively induces neurogenesis in the adult rat hippocampus. J. Neurosci. 20, 2896-903 (2000).

9. Van Praag, H. et al. Functional neurogenesis in the adult hippocampus. Nature 415, 1030-1034 (2002).

10. Stanfield, B.B. & Trice, J.E. Evidence that granule cells generated in the dentate gyrus of adult rats extend axonal projections. Exp. Brain. Res. 72, 399-406 (1988).

11. Perfilieva, E., Risedal, A., Nyberg, J., Johansson, B.B. & Eriksson, P.S. Gender and strain influence on neurogenesis in dentate gyrus of young rats. J. Cereb. Blood. Flow. Metab. 21, 211-217 (2001).

12. Brown, J.C., Pederson, R.A., Jorpes, E. & Mutt, V. Preparation of highly active enterogastrone. Can. J. Physiol. Pharmacol. 47, 113-114 (1969).

13. Buchan, A.M., Polak, J.M., Capella, C., Solcia, E. & Pearse, A.G. Electronimmunocytochemical evidence for the K cell localization of gastric inhibitory polypeptide (GIP) in man. Histochemistry 56, 37-44 (1978).

14. Tseng, C.C., Jarboe, L.A., Landau, S.B., Williams, E.K. & Wolfe, M.M. Glucose-dependent insulinotropic peptide: Structure of the precursor and tissue specific expression in rat. Proc. Natl. Acad. Sci. U S A 90, 1992-1996 (1993).

15. Usdin, T.B., Mezey, E., Button, D.C., Brownstein, M.J. & Bonner, T.I. Gastric inhibitory polypeptide receptor, a member of the secretin-vasoactive intestinal peptide receptor family, is widely distributed in peripheral organs and the brain. Endocrinology 133, 2861-2870 (1993).

16. Higashimoto, Y., Simchock, J. & Liddle, R.A. Molecular cloning of rat glucose-dependent insulinotropic peptide (GIP). Biochim. Biophys. Acta 1132, 72-74 (1992).

17. Sharma, S.K., Austin, C., Howard, A., Lo, G., Nicholl, C.G. & Legon, S. Characterization of rat gastric inhibitory peptide cDNA. J. Mol. Endocrinology 9, 265-272 (1992).

18. Nakajima, T., Nakajima, E., Fukiage, C., Azuma, M. & Shearer, T.R. Differential gene expression in the lens epithelial cells from selenite injected rats. Exp. Eye Res. 74, 231-236 (2002).

19. Yip, R. & Wolfe, M. GIP bolology and fat metabolism. Life Sci. 66, 91-103 (2000).

20. Kogire, M. et al. Effects of gastric inhibitory polypeptide and glucagon on portal venous and hepatic arterial flow in conscious dogs. Dig. Dis. Sci. 3, 1666-1670 (1992).

21. Bollag, R.J. et al. Osteoblast-derived cells express functional glucose-dependent insulinotropic peptide receptors. Endocrinology 141, 1228- 1235 (2000).

22. Hauner, H., Glatting, G., Kaminska, D. & Pfeiffer, E.F. Effects of gastric inhibitory polypeptide on glucose and lipid metabolism of isolated rat adipocytes. Ann. Nutr. Metab. 32, 282-288 (1988).

23. Zhong, Q. et al. Glucose-dependent insulinotropic peptide signaling pathways in endothelial cells. Peptides 21, 1427-1432 (2000).

24. Sherwood, N.M., Krueckl, S.L. & McRory, J.E. The origin and function of the Pituitary Adenylate Cyclase-Activating Polypeptide (PACAP)/glucagon superfamily. Endocrine Rev. 21, 619-670 (2000).

25. Kopin, A.S., Wheeler, M.B. & Leiter, A.B. Secretin: strucure of the precursor and tissue distribution of the mRNA. Proc. Natl. Acad. Sci. U S A 87, 2299-2303 (1990).

26. Buchan, A.M., Ingman-Baker, J., Levy, J. & Brown, J.C. A comparison of the ability of serum and monoclonal antibodies to gastric inhibitory polypeptide to detect immunoreactive cells in the gastroenteropancreatic system of mammals and reptiles. Histochemistry 76, 341-349 (1982).

27. Sjölund, K., Ekelund, M., Håkanson, R., Moody, A.J. & Sundler, F. Gastric inhibitory peptide-like immunoreactivity in glucagon and glicentin cells: properties and origin. An immunocytochemical study using several antisera. J. Histochem. Cytochem. 31, 811-817 (1983).

28. Damholt, A.B., Kofod, H. & Buchan, A.M. Immunocytochemical evidence for a paracrine interaction between GIP and GLP-1 producing cells in canine small intestine. Cell. Tissue Res. 298, 287-293 (1999).

29. Cameron, H.A., Hazel, T.G. & McKay, R.D.Regulation of neurogenesis by growth factors and neurotransmitters. J. Neurobiol. 36, 287-306 (1998).

30. (unpublished data) Åberg, M.A.I. et al. (2002)

31. Chabre, O. et al. Gastric inhibitory polypeptide (GIP) stimulates cortisol secretion, cAMP production and DNA synthesis in an adrenal adenoma responsible for food-dependent Cushing's syndrome. Endocr. Res. 24, 851-856 (1998).

32. Trumper, A., et al. Glucose-dependent insulinotropic polypeptide is a growth factor for beta (INS-1) cells by pleiotropic signaling. Mol. Endocrinol. 15, 1559-70 (2001).

33. Vaudry, D., Gonzalez, B.J., Basille, M., Fournier, A. & Vaudry, H. Neurotrophic activity of pituitary adenylate cyclase-activating polypeptide on rat cerebellar cortex during development. Proc. Natl. Acad. Sci. U S A 96, 9415-9420 (1999).

34. Dicicco-Bloom, E. & Deutsch, P.J. Pituitary adenylate cyclase activating peptide (PACAP) potently stimulates mitosis, neuritogenesis and survival in cultured rat sympathetic neuroblasts. Regul. Pept. 37, 319-325 (1992).

35. Lu, N., Zhou, R. & DiCicco-Bloom, E. Opposing mitogenic regulation by PACAP in sympathetic and cerebral cortical precursors correlates with differential expression of PACAP receptor (PAC1-R) isoforms. J. Neurosci. Res. 53, 651-662 (1998).

36. Suh, J., Lu, N., Nicot, A., Tatsuno, I. & DiCicco-Bloom, E. PACAP is an anti-mitotic signal in developing cerebral cortex. Nat. Neurosci. 4, 123-124 (2001).

37. Waschek, J.A. Vasoactive intestinal peptide: an important trophic factor and developmental regulator? Dev. Neurosci. 17, 1-7 (1995).

38. Pincus, D.W., DiCicco-Bloom, E. & Black, I.B. Trophic mechanisms regulate mitotic neuronal precursors: role of vasoactive intestinal peptide (VIP). Brain Res. 663, 51-60 (1994).

39. Lin, C., Lin, S.C., Chang, C.P. & Rosenfeld, M.G. Pit-1-dependent expression of the receptor for growth hormone releasing factor mediates pituitary cell growth. Nature 360, 765-768 (1992).

40. Mayo, K.E. et al. Dramatic pituitary hyperplasia in transgenic mice expressing a human growth hormone-releasing factor gene. Mol. Endocrinol. 2, 606-612 (1988).

41. Drucker, D.J., Erlich, P., Asa, S.L. & Brubaker, P.L. Induction of intestinal epithelial proliferation by glucagon-like peptide 2. Proc. Natl. Acad. Sci. U S A 93, 7911-7916 (1996).

42. Lovshin, J. & Drucker, D.J. New frontiers in the biology of GLP-2. Regul. Pept. 90, 27-32 (2000).

43. Chomczyski, P. & Sacchi, N. Single-step method of RNA isolation by acid guanidinium thiocyanate-phenol-chloroform extraction. Anal Biochem. 162, 156-159 (1987).

44. Åberg, M.A. et al., Selective introduction of antisense oligonucleotides into single adult CNS progenitor cells using electroporation demonstrates the requirement of STAT3 activation for CNTF-induced gliogenesis. Mol. Cell. Neurosci. 17, 426-443 (2001).

45. Lewis, J.T., Dayanandan, B., Habener, J.F. & Kieffer, T.J. Glucose-dependent insulinotropic polypeptide confers early phase insulin release to oral glucose in rats: demonstration by a receptor antagonist. Endocrinology 141, 3710-6 (2000).

46. Åberg, M.A., Åberg, N.D., Hedbäcker, H., Oscarsson, J. & Eriksson, P.S. Peripheral infusion of IGF-I selectively induces neurogenesis in the adult rat hippocampus. J. Neurosci. 20, 2896-2903 (2000).

47. Dagerlind, Å., Friberg, K., Bean, A.J. & Hökfelt, T. Sensitive mRNA detection using unfixed tissue: combined radioactive and non-radioactive in situ hybridization histochemistry. Histochemistry 98, 39-49 (1992).

48. Peterson, G.L. A simplification of the protein assay method of Lowry et al. which is more generally applicable. Anal. Biochem. 83, 346-356 (1977).

What is Claimed is:

1. A method for prophylaxis and/or treatment of conditions caused or characterized by abnormal loss of cells, comprising: administering to a subject a pharmaceutical composition comprising a compound that when tested in an in vitro proliferation assay has an activity that corresponds to at least about 50% of an activity of SEQ ID NO 2 when tested in a same assay under same conditions .

2. The method according to claim 1, wherein the abnormal loss of cell is a degeneration of neuronal cells, or a loss of astrocytes or oligodendrocytes.

3. The method according to claim 1 , wherein the abnormal loss of cells is caused by traumatic, asphyxia, hypoxic, ischemic, toxic, infectious, degenerative or metabolic insults.

4. The method according to claim 1, wherein the conditions are selected from the group comprising Parkinson's disease, Alzheimer's disease, stroke, multiple sclerosis, asphyxia or hypoxia, heart failure, heart infarction, arthrosis or arthritis, skin disease and burn injuries, diabetes, liver diseases or failure, muscle diseases or damages, pancreatic dysfunction, and diseases caused by prions, such as Creutzfeld-Jacob's disease, scrapie and bovine spongiform encephalitis (BSE).

5. The method according to claim 1, wherein the abnormal loss of cells is caused by insults to the central or peripheral nervous system.

6. The method according to claim 4, wherein the conditions are selected from the group consisting of Parkinson's disease, Alzheimer's disease, stroke, multiple sclerosis, amyotrophic lateral sclerosis, asphyxia or hypoxia, epilepsy, and diseases caused by prions, such as Creutzfeld-Jacob's disease, scrapie and bovine spongiform encephalitis (BSE).

7. The method according to claim 1, wherein the compound has an activity that corresponds to at least about 55%, at least about 60%, at least about 65%, at least about 70%, at least about 75%, at least about 80%, at least about 85%, at least about 90%, at least about 92%, at least about 94%, at least about 96%, at least about 98% or at least about 99% of the activity of SEQ ID NO 2.

8. Claim 8: The method according to claim 1, wherein the compound has an activity that corresponds to at least about 100%, at least about 110%, at least about 120%, at least about 130%, at least about 140%, at least about 150%, at least about 160%, at least about 170%, at least about 180%, at least about 190%, or at least about 200% of the activity of SEQ ID NO 2.

9. The method according to claim 1, wherein the compound is identical to SEQ ID NO 2.

10. The method according to claim 1, wherein the compound has an identity corresponding to at least about 75%, at least about 80%, at least about 85%, at least about 90%, at least about 95%, at least about 96%, at least about 97%, at least about 98% or at least about 99% to SEQ ID NO 2.

11. The method according to claim 1, wherein the compound is similar to SEQ ID NO 2.

12. The method according to claim 1, wherein the compound has a similarity corresponding to at least about 75%, at least about 80%, at least about 85%, at least about 90%, at least about 95%, at least about 96%, at least about 97%, at least about 98% or at least about 99% to SEQ ID NO 2.

13. The method according to claim 1, wherein the compound is SEQ ID NO 2, analogues or fragments thereof.

14. A compound that, when tested in an in vitro proliferation assay, has an activity that corresponds to at least about 50% of an activity of SEQ ID NO 2 when tested in a same assay under same conditions with a proviso that the compound is not SEQ ID NO 2 or basic fibroblast growth factor BFGF.

15. A compound according to claim 14 for medicinal use.

16. A compound according to claim 14, said compound is administered to a subject for a prophylaxis and/or treatment of conditions caused by abnormal loss of cells.

17. A method comprising: administering to a subject an antagonist to GIP for a prophylaxis and/or treatment of conditions caused or characterized by hyperproliferation of cells.

18. A method comprising: administering to a subject an antibody against GIP for the prophylaxis and/or treatment of conditions caused or characterized by hyperproliferation of cells.

19. A method comprising: administering to a subject a pharmaceutical composition comprising an antagonist to the GIP receptor for a prophylaxis and/or treatment of conditions caused or characterized by hyperproliferation of cells.

20. The method according to claim 17, wherein the conditions are selected from neoplastic or cancer diseases such as, e. g., melanoma, non-small-cell lung cancer, small- cell lung cancer, lung cancer, hepatocarcinoma, retioblastoma, astrocytoma, glioblastoma, leukemia, neuroblastoma, pre-neoplastic lesions such as adenomatous hyperplasia and prostatic intraepithelial neoplasia, carcinoma in situ, cancer in the gum, tongue, head, neck, breast, pancreas, prostate, kidney, liver, bone, thyroid, testicle, ovary, mesothelia, cervix, gastrointestinal tract, lymphom, brain, colon, sarcoma and bladder.

21. The method according to claim 17, wherein the conditions are selected from tumor- associated diseased, rheumatoid arthritis, inflammatory bowel disease, osteoarthritis, leiomyomas, adenomas, lipomas. hemagioomas, fibromas, vascular occlusion, retenosis, atherosclerosis, oral hairy leukoplasia, benign prostatic hyperplasia, or psoriasis.

22. A method for prophylaxis or treatment of overweight and/or obesity comprising:
administering to a subject a pharmaceutical composition comprising a compound which when given intraventricularly in the brain of rats, followed by the recordation of the weight of each rat, the compound has an activity in reducing weight gain that corresponds to at least about 50% of the activity of SEQ ID NO 2 or SEQ ID NO 4 when tested in a same assay under same conditions using a compound having SEQ ID NO 2 or SEQ ID NO 4 as a control.

23. The method according to claim 22, wherein the pharmaceutical composition further comprises a carrier allowing the transport of the compound across the blood brain barrier.

24. The method according to claim 22, wherein the compound has an activity that corresponds to at least about 55%, at least about 60%, at least about 65%, at least about 70%, at least about 75%, at least about 80%, at least about 85%, at least about 90%, at least about 92%, at least about 94%, at least about 96%, at least about 98% or at least about 99% of the activity of SEQ ID NO 2 or SEQ ID NO 4.

25. The method according to claim 22, wherein the compound has an activity that corresponds to at least about 100%, at least about 110%, at least about 120%, at least about 130%, at least about 140%, at least about 150%, at least about 160%, at least about 170%, at least about 180%, at least about 190%, or at least about 200% of the activity of SEQ ID NO 2 or SEQ ID NO 4.

26. The method according to claim 22, wherein the compound is identical to SEQ ID NO 2 or SEQ ID NO 4.

27. The method according to claim 22, wherein the compound has an identity corresponding to at least about 75%, at least about 80%, at least about 85%, at least about 90%, at least about 95%, at least about 96%, at least about 97%, at least about 98% or at least about 99% to SEQ ID NO 2 or SEQ ID NO 4.

28. The method according to claim 22, wherein the compound is similar to SEQ ID NO 2 or SEQ ID NO 4.

29. The method according to claim 22, wherein the compound has a similarity corresponding to at least about 75%, at least about 80%, at least about 85%, at least about 90%, at least about 95%, at least about 96%, at least about 97%, at least about 98% or at least about 99% to SEQ ID NO 2 or SEQ ID NO 4.

30. The method according to claim 22, wherein the compound is SEQ ID NO 2 or SEQ ID NO 4, analogues or fragments thereof.

31. A compound having an activity in reducing weight gain that corresponds to at least about 50% of an activity of SEQ ID NO 2 or SEQ ID NO 4 when the compound is given intraventricularly in the brain of rats, followed by the recordation of the weight of each rat when tested in a same assay under same conditions.

32. A compound according to claim 31 for medicinal use.

33. A compound according to claim 32, the compound provided for a prophylaxis and/or treatment of overweight and/or obesity.

34. A method of prophylaxis and/or treatment of overweight and/or obesity, the method comprising administering to a subject a pharmaceutical composition comprising a compound according to claim 31 by an intraventricular route.

35. A cosmetic method for reducing body weight, the method comprising administering to a subject a composition comprising a compound according to claim 31.

36. The method comprising: administering to a subject a pharmaceutical composition comprising an antagonist to GIP for a prophylaxis and/or treatment of conditions caused or characterized by abnormally low body weight.

37. The method comprising: administering to a subject an antibody against GIP for a prophylaxis and/or treatment of conditions caused or characterized by abnormally low body weight.

38. A method comprising: administering to a subject a pharmaceutical composition comprising an antagonist to the GIP receptor for a prophylaxis and/or treatment of conditions caused or characterized by abnormally low body weight.

39. The method according to claim 36, wherein the condition is selected from anorexia, cachexia, AIDS-or cancer-related wasting, and failure to thrive syndrome in newborn and young children.

40. A pharmaceutical composition comprising a compound according to claim 14 together with one or more pharmaceutical acceptable excipients.

41. The method comprising: providing a compound having SEQ ID NO 2 or analogues, functional analogues or fragments thereof for a manufacture of a pharmaceutical composition for prophylaxis and/or treatment of depression and/or mood disorders.

42. A method for determining an abnormal level of GIP in the brain of a mammal.

43. A method according to claim 42 for diagnosis, disease monitoring and/or therapeutic monitoring of a disease characterized by an abnormal amount of GIP in the brain.

44. A method according to claim 42, wherein the level of GIP in the brain of a subject is low compared to a healthy subject.

45. A method according to claim 42, wherein the level of GIP in the brain of a subject is high compared to a healthy subject.

46. The method according to claim 18, wherein the conditions are selected from neoplastic or cancer diseases such as, e. g., melanoma, non-small-cell lung cancer, small- cell lung cancer, lung cancer, hepatocarcinoma, retioblastoma, astrocytoma, glioblastoma, leukemia, neuroblastoma, pre-neoplastic lesions such as adenomatous hyperplasia and prostatic intraepithelial neoplasia, carcinoma in situ, cancer in the gum, tongue, head, neck, breast, pancreas, prostate, kidney, liver, bone, thyroid, testicle, ovary, mesothelia, cervix, gastrointestinal tract, lymphom, brain, colon, sarcoma and bladder.

47. The method according to claim 18, wherein the conditions are selected from tumor- associated diseased, rheumatoid arthritis, inflammatory bowel disease, osteoarthritis, leiomyomas, adenomas, lipomas. hemagioomas, fibromas, vascular occlusion, retenosis, atherosclerosis, oral hairy leukoplasia, benign prostatic hyperplasia, or psoriasis.

48. The method according to claim 19, wherein the conditions are selected from neoplastic or cancer diseases such as, e. g., melanoma, non-small-cell lung cancer, small- cell lung cancer, lung cancer, hepatocarcinoma, retioblastoma, astrocytoma, glioblastoma, leukemia, neuroblastoma, pre-neoplastic lesions such as adenomatous hyperplasia and prostatic intraepithelial neoplasia, carcinoma in situ, cancer in the gum, tongue, head, neck, breast, pancreas, prostate, kidney, liver, bone, thyroid, testicle, ovary, mesothelia, cervix, gastrointestinal tract, lymphom, brain, colon, sarcoma and bladder.

49. The method according to claim 19, wherein the conditions are selected from tumor- associated diseased, rheumatoid arthritis, inflammatory bowel disease, osteoarthritis, leiomyomas, adenomas, lipomas. hemagioomas, fibromas, vascular occlusion, retenosis, atherosclerosis, oral hairy leukoplasia, benign prostatic hyperplasia, or psoriasis.

50. The method according to claim 37, wherein the condition is selected from anorexia, cachexia, AIDS-or cancer-related wasting, and failure to thrive syndrom in newborn and young children.

51. The method according to claim 38, wherein the condition is selected from anorexia, cachexia, AIDS-or cancer-related wasting, and failure to thrive syndrom in newborn and young children.

52. A pharmaceutical composition comprising a compound according to claim 31 together with one or more pharmaceutical acceptable excipients.

53. A pharmaceutical composition for prophylaxis and/or treatment of conditions caused or characterized by abnormal loss of cells, comprising:
a compound that when tested in an in vitro proliferation assay has an activity that corresponds to at least about 50% of an activity of SEQ ID NO 2 when tested in a same assay under same conditions.

54. The composition according to claim 53, wherein the abnormal loss of cell is a degeneration of neuronal cells, or a loss of astrocytes or oligodendrocytes.

55. The composition according to claim 53, wherein the abnormal loss of cells is caused by traumatic, asphyxia, hypoxic, ischemic, toxic, infectious, degenerative or metabolic insults.

56. The composition according to claim 53, wherein the conditions are selected from the group comprising Parkinson's disease, Alzheimer's disease, stroke, multiple sclerosis, asphyxia or hypoxia, heart failure, heart infarction, arthrosis or arthritis, skin disease and burn injuries, diabetes, liver diseases or failure, muscle diseases or damages, pancreatic dysfunction, and diseases caused by prions, such as Creutzfeld-Jacob's disease, scrapie and bovine spongiform encephalitis (BSE).

57. The composition according to claim 53, wherein the abnormal loss of cells is caused by insults to the central or peripheral nervous system.

58. The composition according to claim 56, wherein the conditions are selected from the group consisting of Parkinson's disease, Alzheimer's disease, stroke, multiple sclerosis, amyotrophic lateral sclerosis, asphyxia or hypoxia, epilepsy, and diseases caused by prions, such as Creutzfeld-Jacob's disease, scrapie and bovine spongiform encephalitis (BSE).

59. The composition according to claim 53, wherein the compound has an activity that corresponds to at least about 55%, at least about 60%, at least about 65%, at least about 70%, at least about 75%, at least about 80%, at least about 85%, at least about 90%, at least about 92%, at least about 94%, at least about 96%, at least about 98% or at least about 99% of the activity of SEQ ID NO 2.

60. The composition according to claim 53, wherein the compound has an activity that corresponds to at least about 100%, at least about 110%, at least about 120%, at least about 130%, at least about 140%, at least about 150%, at least about 160%, at least about 170%, at least about 180%, at least about 190%, or at least about 200% of the activity of SEQ ID NO 2.

61. The composition according to claim 53, wherein the compound is identical to SEQ ID NO 2.

62. The composition according to claim 53, wherein the compound has an identity corresponding to at least about 75%, at least about 80%, at least about 85%, at least about 90%, at least about 95%, at least about 96%, at least about 97%, at least about 98% or at least about 99% to SEQ ID NO 2.

63. The composition according to claim 53, wherein the compound is similar to SEQ ID NO 2.

64. The composition according to claim 53, wherein the compound has a similarity corresponding to at least about 75%, at least about 80%, at least about 85%, at least about 90%, at least about 95%, at least about 96%, at least about 97%, at least about 98% or at least about 99% to SEQ ID NO 2.

65. The composition according to claim 53, wherein the compound is SEQ ID NO 2, analogues or fragments thereof.
